(19) United States
(12) Patent Application Publication
(10) Pub. No.: US 2003/0036564 A1
(43) Pub. Date: Feb. 20, 2003

(54) Control of lyme disease spirochete

(76) Inventors: Jeff N. Borchert, Ft. Collins, CO (US); Richard M. Poche, Wellington, CO (US)

Correspondence Address: Dean P. Edmundson, P.O. Box 179, Burton, TX, 77835, US

(21) Appl. No.: 10/215,568

(22) Filed: Aug. 8, 2002

Related U.S. Application Data
(60) Provisional application No. 60/310,884, filed on Aug. 8, 2001.

(51) Int. Cl. A61K 31/16; A23K 1/165

(52) U.S. Cl. 514/627; 514/628; 424/442

(57) ABSTRACT

A method is described for controlling the spread of Lyme disease spirochete from rodents which have been infected. The method involves orally administering to the rodents a composition which includes an antibiotic, e.g. chloramphenicol, thiamphenicol, florfenicol, or a salt or derivative thereof, or mixtures of antibiotics, capable of killing the spirochete. Bait compositions are described which include an antibiotic. The bait compositions may be solid or liquid.

### CROSS-REFERENCE TO RELATED APPLICATION

This application is based upon, and claims the benefit of, our U.S. Provisional Application No. 60/310,884, filed Aug. 8, 2001.

### FIELD OF THE INVENTION

This invention relates to methods and techniques for controlling Lyme disease spirochete in rodents.

### BACKGROUND OF THE INVENTION

Lyme disease has increased about 25-fold since national surveillance began in 1982. The average number of human cases reported from 1993-1997 was 12,500, according to CDC (Center for Disease Control, 1999). The CDC reports that Lyme disease accounts for more than 95% of all reported vector-borne illnesses in the US and more than 128,000 human cases have been reported to health authorities. The disease is reportedly primarily localized to states in the northeastern, mid-Atlantic, and upper north-central regions and to several areas in northwestern California. In the United States, two ticks of the Ixodes genus, Ixodes scapularis in the East and Ixodes pacificus in the West commonly carry the spirochete that causes the disease and are in a two-year enzootic cycle with rodents (primarily Peromyscus leucopus) and deer (primarily Odocoileus virginianus) .

U.S. Pat. No. 5,932,437 describes that spirochete infection in rodents can be cured by the use of baits which have been treated with an antibiotic.

### SUMMARY OF THE INVENTION

In accordance with the present invention, there is provided a method for interrupting the enzootic cycle to thereby control the spread of Lyme disease spirochete using the antibiotic chloramphenicol and its derivatives, thiamphenicol, florfenicol, or a salt or derivative thereof. The present invention also provides bait compositions comprised of one or more of such antibiotics, alone or in combination with the antibiotics described in U.S. Pat. No. 5,932,437, incorporated herein by reference.

In one embodiment of the invention, the method involves orally administering to rodents in the wild a composition containing an effective amount of one or more of the above-described antibiotics which are capable of killing the spirochete.

Using the technique of this invention, a bait composition which has been treated to include one or more of the above antibiotics can be placed in the wild where rodents can feed on it. Ingestion of the composition by rodents which have been infected results in elimination of the spirochete bacterium.

Other features and advantages of the invention will be apparent from the following detailed description.

### DETAILED DESCRIPTION OF THE INVENTION

The method and technique of this invention involve preparing a bait composition which can be placed in the wild in areas where rodents are located and which may be infected with the Lyme disease spirochete. The bait should be provided in amounts of about 200 grams in protected bait stations or in places which are protected from children, pets and wildlife other than rodents.

Preferred compositions include a palatable feed mixture containing at least about 500 ppm of a suitable antibiotic such as chloramphenicol, thiamphenicol, florfenicol, or a salt or derivative thereof, or mixtures of such antibiotics, or mixtures of any of the foregoing antibiotics with the antibiotics described in U.S. Pat. No. 5,932,437, e.g. tetracycline, doxycycline, erythromycin, azithromycin, minocycline, metronidazole, or a salt or derivative thereof.

The amount of antibiotic in the composition is preferably in the range of about 100 ppm to about 1500 ppm or even greater concentrations if necessary, depending upon the particular antibiotic(s) used. The bait may comprise a water-based composition with the antibiotic dissolved in the water or suspended in a palatable solvent.

### EXAMPLE 1

A bait composition was prepared which included the following ingredients: chloramphenicol, commercially prepared food grade corn meal as a diet carrier, corn oil and confectioner's sugar. The diet was formulated at a theoretical concentration of 500 mg/kg chloramphenicol, 1% by weight corn oil, and 1% by weight sugar. The chloramphenicol and confectioner's sugar were mixed thoroughly in a plastic bag. The corn meal was placed under a Hobart mixer and mixed at a moderate speed. The corn oil was slowly added, followed by the antibiotic/sugar mixture. The composition was allowed to mix for approximately 30 minutes. The concentration of chloramphenicol in the final composition was 500 ppm.

### EXAMPLE 2

A bait composition was prepared using the same ingredients and procedure as described in Example 1, except that the amounts of sugar and chloramphenicol were adjusted to obtain a concentration of antibiotic in the final composition of 1000 ppm.

### EXAMPLE 3

A bait composition was prepared using the same ingredients and procedure as described in Example 1, except that thiamphenicol was added instead of chloramphenicol to obtain a concentration of antibiotic in the final composition of 500 ppm.

### EXAMPLE 4

The diet compositions described in Examples 1-3 were fed separately on a no-choice basis to separate groups of laboratory mice (Swiss-Webster) infected with the Lyme disease spirochete Borrelia burgdorferi. Six mice were used for each test composition. The mice had all been previously infected with B. burgdorferi strain B31 by subcutaneous injection followed by a minimum 21-day incubation period. To confirm that the mice were positive for the spirochete, a modified ear punch biopsy was performed (Sinsky and Piesman, 1989, J. of Clin. Micro. 27:1723-1727, CDC 1997) on each mouse and the biopsy incubated in a modified BSK-H media for 7 days and viewed under dark field microscopy. The prepared diets were fed to individually housed mice on a no-choice basis and water was offered ad libitum. Consumption was recorded each day and additional feed was added. Diet was presented to the mice for seven consecutive days. On the day following the last day feed was offered, mice were anesthetized with Halothane and ear punch biopsy was performed. The biopsies were incubated in modified BSK-H media for seven days at 34±1° C. Dark field microscopy was then performed on an American Optics 1031 microscope with an American Optics dark field 1096 condenser to determine the presence of spirochetes. Cultures were considered positive if spirochete growth occurred within five weeks of inoculation (Wilske and Preac-Mursic, Aspects of Lyme Borreliosis, Springer-Verlag, New York 1993), viewing the cultures at seven-day increments. A slide was considered negative if no spirochetes were found after searching 100 fields at 400× (Ginsberg and Ewing, J. of Med. Entomology, 26: 183-189, 1989). The results are shown in the following tables.

TABLE 1
TREATMENT LEVEL 1 RESULTS
CHLORAMPHENICOL 500 μg/g

| | |
|---|---|
| MEAN BAIT CONSUMPTION (G) | 41.3 ± 4.3 |
| MEAN CHLORAMPHENICOL INTAKE | 2.9 ± 0.3 |
| PER DAY (mg) | |
| NUMBER AND PERCENTAGE OF MICE | 10/10 |
| TESTING NEGATIVE FOR SPIROCHETE | (100%) |

TABLE 2
TREATMENT LEVEL 2 RESULTS
CHLORAMPHENICOL 1000 μg/g

| | |
|---|---|
| MEAN BAIT CONSUMPTION (G) | 42.2 ± 5.5 |
| MEAN CHLORAMPHENICOL INTAKE | 6.1 ± 0.8 |
| PER DAY (mg) | |
| NUMBER AND PERCENTAGE OF MICE | 10/10 |
| TESTING NEGATIVE FOR SPIROCHETE | (100%) |

TABLE 3
TREATMENT LEVEL 3 RESULTS
THIAMPHENICOL 1000 μg/g

| | |
|---|---|
| MEAN BAIT CONSUMPTION (G) | 39.9 ± 20.3 |
| MEAN THIAMPHENICOL INTAKE | 2.9 ± 1.4 |
| PER DAY (mg) | |
| NUMBER AND PERCENTAGE OF MICE | 10/10 |
| TESTING NEGATIVE FOR SPIROCHETE | (100%) |

What is claimed is:

1. A method for controlling the spread of Lyme disease spirochete from rodents which have been infected therewith, the method comprising the step of orally administering to said rodents in the wild a composition containing an effective amount of an antibiotic capable of killing said spirochete; wherein said antibiotic is selected from the group consisting of chloramphenicol, thiamphenicol, florfenicol, or a salt or derivative thereof.

2. A method in accordance with claim 1, wherein said composition additionally comprises grain and a sugar.

3. A method in accordance with claim 1, wherein said antibiotic comprises chloramphenicol or a salt or derivative thereof.

4. A method in accordance with claim 1, wherein said antibiotic is present in said composition in an amount of at least about 0.025% by weight.

5. A method in accordance with claim 1, wherein said composition is aqueous.

6. A method for killing Lyme disease spirochete present in rodents in the wild, comprising the step of feeding said rodents a bait composition comprising an antibiotic capable of killing said spirochete; wherein said antibiotic is selected from the group consisting of chloramphenicol, thiamphenicol, florfenicol, or a salt or derivative thereof.

7. A method in accordance with claim 6, wherein said composition is aqueous.

8. A method in accordance with claim 6, wherein said composition additionally comprises grain and a sugar.
